(19) United States
(12) Patent Application Publication
(10) Pub. No.: US 2021/0030579 A1
(43) Pub. Date: Feb. 4, 2021

(54) ARTHRITIS CORRECTION AND FIXATION DEVICE

(71) Applicant: Orthomis Medical, Inc., Taipei City (TW)

(72) Inventors: Meng-Shin YEN, Taipei City (TW); Cheng-Yu LIU, Taipei City (TW); Chung-Ping CHUANG, Taipei City (TW)

(21) Appl. No.: 16/526,512

(22) Filed: Jul. 30, 2019

(51) Int. Cl. A61F 5/058; A61B 17/72; A61B 17/66; A61F 5/10; A61F 5/01

(52) U.S. Cl. CPC A61F 5/05875; A61B 17/7291; A61B 17/86; A61F 5/10; A61F 5/0118; A61B 17/66

(57) ABSTRACT

An arthritis correction and fixation device is used to connect distal and middle phalanges of a finger. The arthritis correction and fixation device includes a first unit, a second unit and a connection unit. The first unit connects with the distal phalange and has a first threaded portion and a first connecting portion. The second unit connects with the middle phalange and has a second threaded portion and a second connecting portion. The first connecting portion and the second connecting portion are connected through the connection unit, so that the first unit and the second unit can actuate relative to each other. When the first threaded portion is rotated, the first connecting portion drives the second unit to move together in a direction away from the first threaded portion, thereby changing a distance between the distal and middle phalanges.

### BACKGROUND

### Field

The invention relates to finger joint; in particular, to an arthritis correction and fixation device.

### Description of the Related Art

The joint is the connecting area of the two bones of the human body, which is in charge of many movements of the human body (such as knee flexion, fist clenching, etc.). Among the joints responsible for exercise, most of the joints are covered by an articular capsule, which looks like a wrap around two bones, with cartilage and synovial fluid. The cartilage provides cushioning for the two bone connections. Synovial fluid is like a lubricating fluid, which makes the bones move smoothly.

Once cartilage wear or synovial secretion is abnormal due to various reasons, pain, swelling, fever, stiffness and the like are generated, which are typical symptoms of arthritis. There are many types of arthritis, such as rheumatoid arthritis, degenerative arthritis, gouty arthritis and the like.

When the patient's fingers suffer from arthritis, the cartilage of the knuckle has worn out, causing the distal phalange and the middle phalange to rub against each other and causing pain, which causes the patient's fingers to feel uncomfortable during movement. Therefore, an appropriate finger correction and fixation device is needed to assist the patient.

### SUMMARY

Therefore, the invention provides an arthritis correction and fixation device to solve the above-mentioned problems occurred in the prior arts.

An embodiment of the invention is an arthritis correction and fixation device. In this embodiment, the arthritis correction and fixation device is used to connect distal and middle phalanges of a finger. The arthritis correction and fixation device includes a first unit, a second unit and a connection unit. The first unit connects with the distal phalange and has a first threaded portion and a first connecting portion. The second unit connects with the middle phalange and has a second threaded portion and a second connecting portion. The first connecting portion and the second connecting portion are connected through the connection unit, so that the first unit and the second unit can actuate relative to each other. When the first threaded portion is rotated, the first connecting portion drives the second unit to move together in a direction away from the first threaded portion, thereby changing a distance between the distal and middle phalanges. The first unit further includes a screw disposed in the first threaded portion and engaged with the first connecting portion, and rotating directions of the screw and the first threaded portion are opposite to each other. When the first threaded portion is rotated in a first rotational direction, the screw rotates in a second rotational direction opposite to the first rotational direction to move away from the first threaded portion, and the screw drives the first connecting portion engaged with the screw and the second unit connected to the first connecting portion both move away from the first threaded portion, thereby increasing the distance between the distal phalange and the middle phalange.

In an embodiment, the first connecting portion has an accommodating space for accommodating the second connecting portion.

In an embodiment, the first connecting portion has a first through hole and the second connecting portion has a second through hole; when the second connecting portion is accommodated in the accommodating space of the first connecting portion, the first through hole and the second through hole are aligned with each other, and the connection unit passes through the first through hole and the second through hole to connect the first connecting portion and the second connecting portion.

Compared to the prior art, when the patient's finger is suffering from arthritis, the arthritis correction and fixation device of the invention can be disposed in the patient's finger and connected between the distal phalange and the middle phalange, and the joint distraction method can be used according to the actual needs of the patient to adjust the distance between the distal phalange and the middle phalange, so that the distal phalange and the middle phalange can be relatively open to prevent the distal phalange and the middle phalange from rubbing against each other, and the pain caused by the patient's finger movement can be effectively reduced.

The advantage and spirit of the invention may be understood by the following detailed descriptions together with the appended drawings.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 illustrates an exploded view showing the screw 10, the fixing portion 11 and the connecting seat 12.

FIG. 2A and FIG. 2B illustrate a schematic view and a cross-sectional view showing that the screw 10 is engaged with the connecting seat 12 through the fixing portion 11 respectively.

FIG. 3 illustrates an exploded view of the first connecting portion 1 and the first threaded portion 2.

FIG. 4 illustrates a schematic view showing the first threaded portion 2 being set on the screw 10 of the first connecting portion 1 to form the first unit 3.

FIG. 5 illustrates an exploded view showing the first unit 3 and the second unit 4 connected through the connection unit 5.

FIG. 6 illustrates a schematic view showing the arthritis correction and fixation device 6.

FIG. 7 and FIG. 8 illustrate schematic diagrams of increasing the distance between the distal phalange FB1 and the middle phalange FB2 through the arthritis correction and fixation device 6 disposed between the distal phalange FB1 and the middle phalange FB2.

FIG. 9 illustrates a schematic diagram of further adjusting the distance between the distal phalange FB1 and the middle phalange FB2 through the auxiliary device 9.

FIG. 10A and FIG. 10B illustrate a side view and a top view of FIG. 9 respectively.

FIG. 11 illustrates a flowchart of the arthritis correction and fixation device operating method according to another embodiment of the invention.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

An embodiment of the disclosure is an arthritis correction and fixation device.

In this embodiment, the arthritis correction and fixation device is disposed in the patient's finger and connected between a distal phalange and a middle phalange of the finger. It is noted the device is not limited to this joint connection.

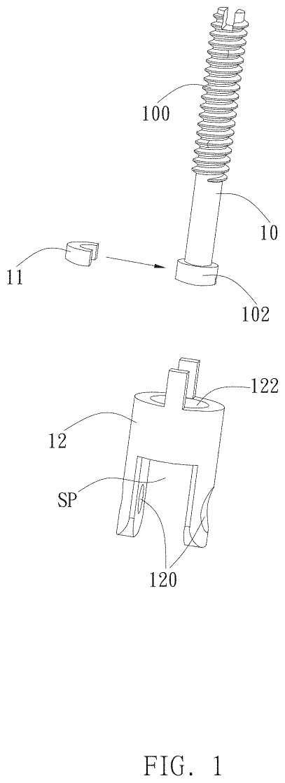

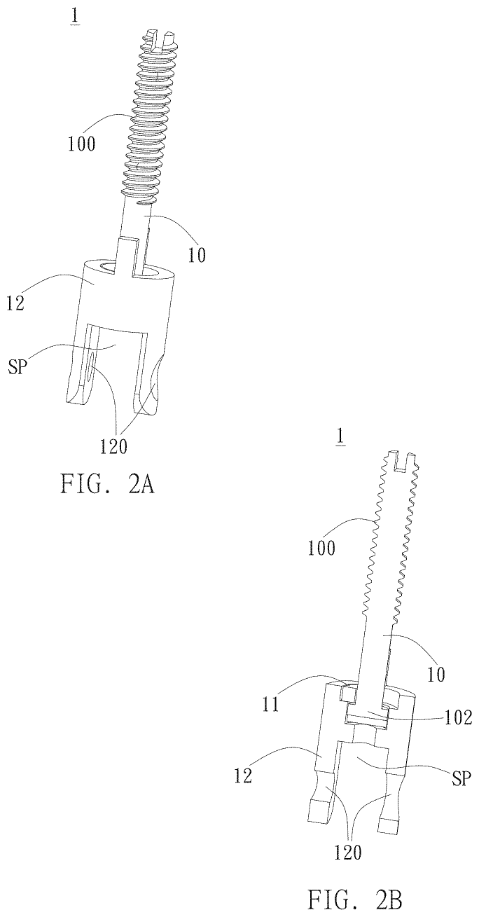

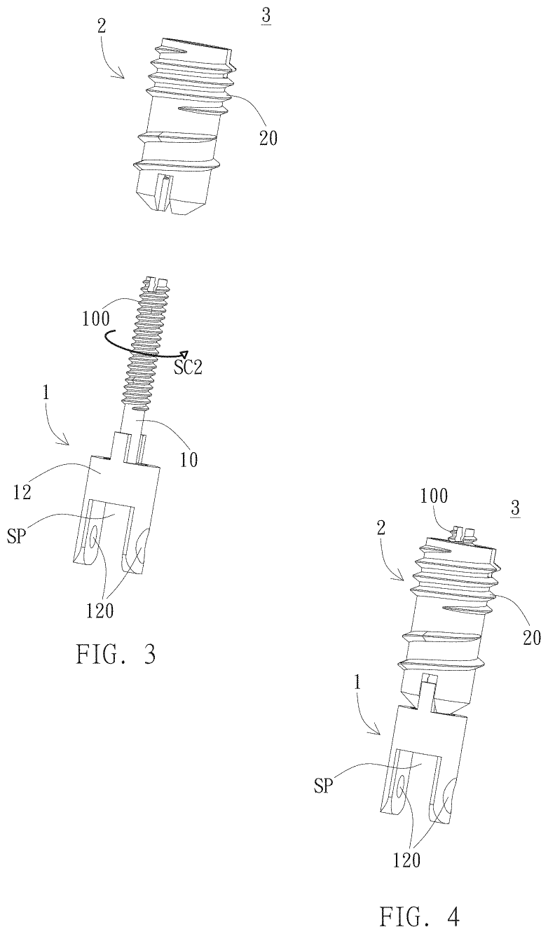

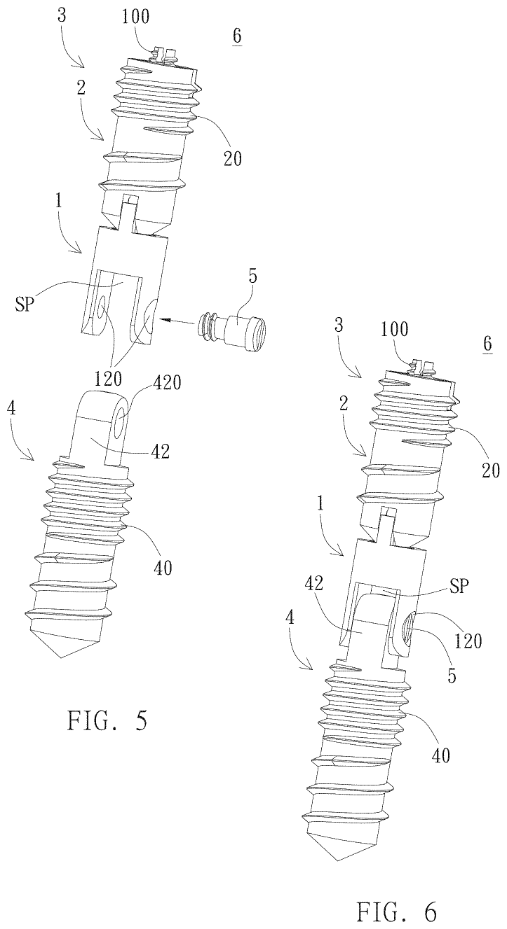

FIG. 1 to FIG. 5 illustrate exploded schematic views showing the components of the arthritis correction and fixation device 6, respectively. FIG. 6 illustrates a schematic view showing the arthritis correction and fixation device 6. In particular, FIG. 1 illustrates an exploded view showing the screw 10, the fixing portion 11 and the connecting seat 12. FIG. 2A and FIG. 2B illustrate a schematic view and a cross-sectional view showing that the screw 10 is engaged with the connecting seat 12 through the fixing portion 11 respectively. FIG. 3 illustrates an exploded view of the first connecting portion 1 and the first threaded portion 2. FIG. 4 illustrates a schematic view showing the first threaded portion 2 being set on the screw 10 of the first connecting portion 1 to form the first unit 3. FIG. 5 illustrates an exploded view showing the first unit 3 and the second unit 4 connected through the connection unit 5. FIG. 6 illustrates a schematic view showing the arthritis correction and fixation device 6.

First, it can be seen from FIG. 6 that the arthritis correction and fixation device 6 can include a first unit 3, a second unit 4 and a connection unit 5. The first unit 3 and the second unit 4 are connected to each other through the connection unit 5, so that the first unit 3 and the second unit 4 can be actuate relative to each other. The first unit 3 can include a first connecting portion 1 and a first threaded portion 2, and the first threaded portion 2 has a thread 20.

Next, it can be seen from FIG. 1 to FIG. 4 that the first connecting portion 1 in the first unit 3 can include a screw 10, a fixing portion 11 and a connecting base 12. The screw 10 can include a threaded portion 100 and a bottom portion 102, and the threaded portion 100 and the bottom portion 102 are connected to each other. The connecting base 12 can include a first through hole 120, a groove 122 and an accommodating space SP. The groove 122 is disposed above the connecting base 12 for accommodating the fixing portion 11 and the bottom portion 102 of the screw 10 to engage the bottom portion 102 of the screw 10 with the groove 122 of the connecting base 12 through the fixing portion 11. The accommodating space SP is located below the connecting base 12, and the first through hole 120 is disposed corresponding to the accommodating space SP.

After the screw 10 is engaged with the connecting base 12 through the fixing portion 11, the first threaded portion 2 can be set on the screw 10 of the first connecting portion 1 to form the first unit 3, as shown in FIG. 3 and FIG. 4. It should be noted that the screw 10 is disposed in the first threaded portion 2 and is engaged with the first connecting portion 12, and rotating directions of the threaded portion 100 of the screw 10 and the first threaded portion 2 are opposite to each other, for example, rotating clockwise and counterclockwise respectively, but not limited to this.

As shown in FIG. 5, the second unit 4 can have a second threaded portion 40 and a second connecting portion 42, and the second threaded portion 40 and the second connecting portion 42 are connected to each other or integrally formed. The second connecting portion 42 has a second through hole 420. When the second connecting portion 42 is accommodated in the accommodating space SP of the first connecting portion 1, the first through hole 120 of the first connecting portion 1 and the second through hole 420 of the second connecting portion 42 can be aligned with each other, and the connection unit 5 simultaneously passes through the first through hole 120 and the second through hole 420 to connect the first connecting portion 12 and the second connecting portion 42, so that the first unit 3 and the second unit 4 can be actuate relative to each other, but not limited to this.

In fact, the connection unit 5 can be a pivoting shaft, so that the first unit 3 and the second unit 4 connected to each other through the connection unit 5 can have a specific angle with respect to each other with the connection unit 5 as an axis, but not limited to this.

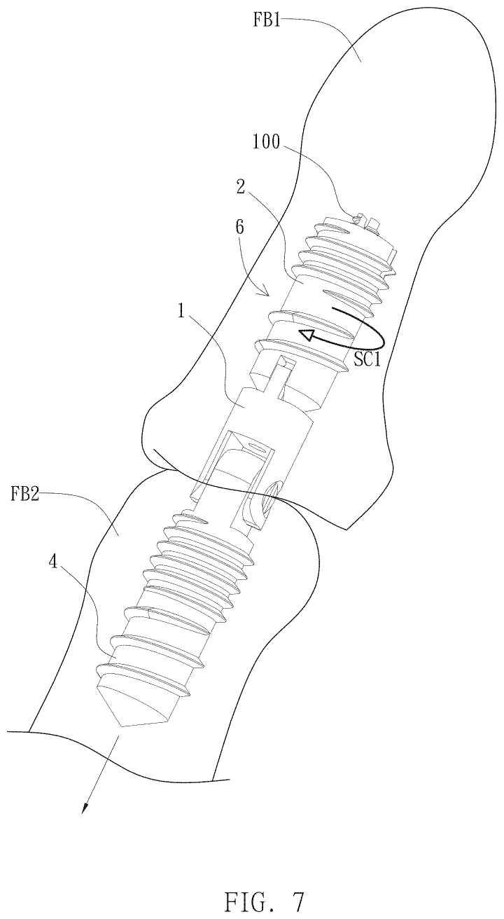

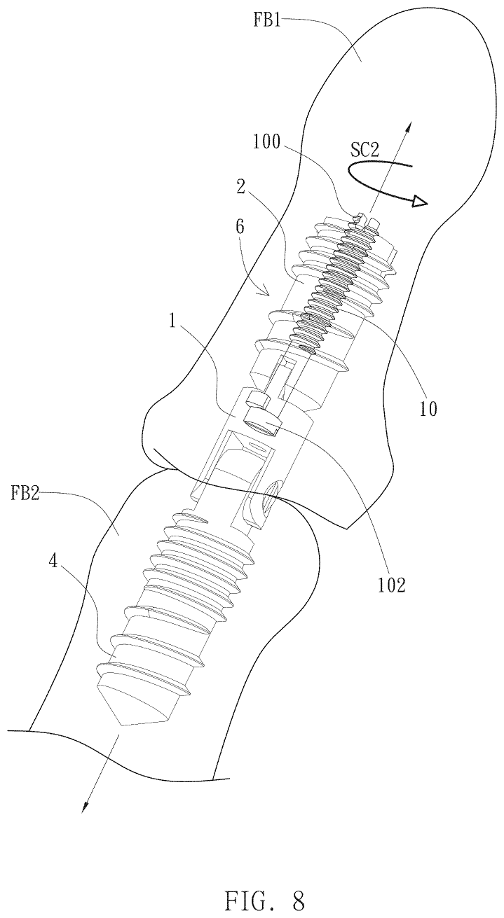

FIG. 7 and FIG. 8 illustrate schematic diagrams of increasing the distance between the distal phalange FB1 and the middle phalange FB2 through the arthritis correction and fixation device 6 disposed between the distal phalange FB1 and the middle phalange FB2.

As shown in FIG. 7 and FIG. 8, the first threaded portion 2 of the first unit 3 is connected to the distal phalange FB1 and the second unit 4 is connected to the middle phalange FB2. When the first threaded portion 2 of the first unit 3 is rotated in a first rotational direction (for example, the clockwise direction) SC1, the rotational direction of the threaded portion 100 of the screw 10 located in the first threaded portion 2 is opposite to the rotation direction of the first threaded portion 2; that is to say, the threaded portion 100 of the screw 10 is rotated in a second rotational direction (for example, counterclockwise direction) SC2 to move away from the first threaded portion 2 to drive the engaged first connecting portion 1 and the second unit 4 connected to the first connecting portion 1 both move away from the first threaded portion 2. By doing so, the joint distraction method is used to drive the middle phalange FB2 move away from the distal phalange FB1 to increase the distance between the distal phalange FB1 and the middle phalange FB2 to reduce the possibility of friction between the distal phalange FB1 and the middle phalange FB2. Therefore, the phenomenon that the distal phalange FB1 and the middle phalange FB2 rub against each other can be effectively improved to slow down the pain caused by arthritis.

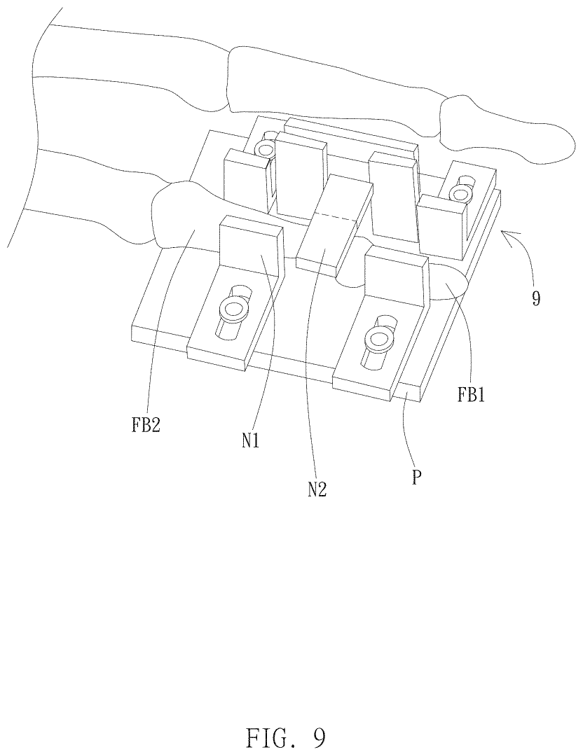

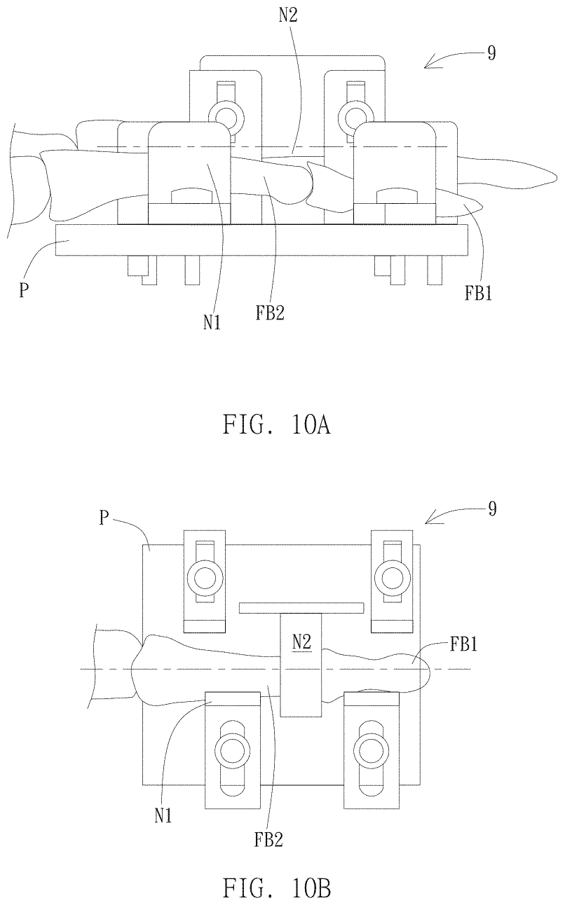

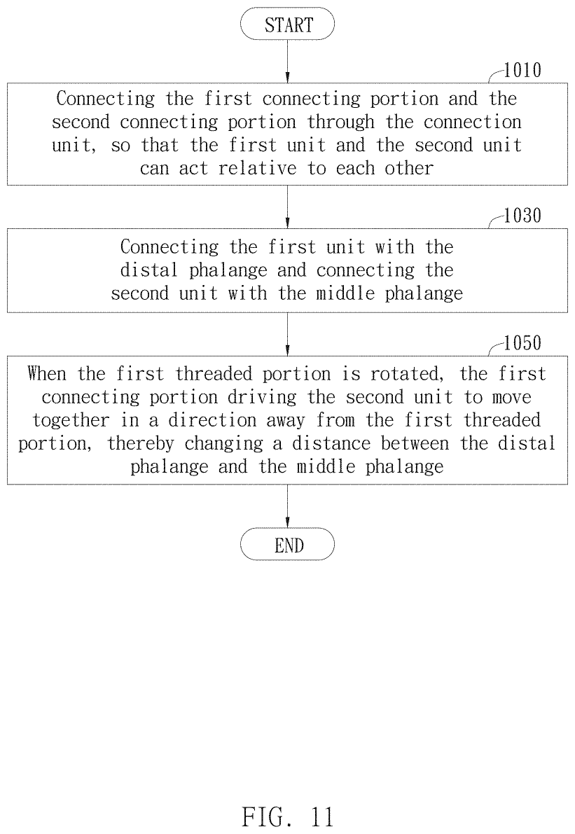

In addition, as shown in FIG. 9, embodiments of the device can further assist the distance adjustment between the distal phalange FB1 and the middle phalange FB2 through the auxiliary device 9. For example, the auxiliary device 9 can include a substrate P, an auxiliary unit N1 and a control unit N2, and the auxiliary unit N1 and the control unit N2 are disposed on the substrate P. When the distal phalange FB1 and the middle phalange FB2 are placed on the space formed by the plurality of auxiliary units N1 on the substrate P, the control unit N2 can be disposed above the junction of the distal phalange FB1 and the middle phalange FB2 (e.g., the finger joint) to control the operation of the arthritis correction and fixation device disposed between the distal phalange FB1 and the middle phalange FB2, but not limited to this. FIG. 10A and FIG. 10B illustrate a side view and a top view of FIG. 9 respectively.

Another embodiment of the invention is an arthritis correction and fixation device operating method.

In one embodiment, the arthritis correction and fixation device operating method is used to operate an arthritis correction and fixation device to connect a distal phalange and a middle phalange of a finger.

The arthritis correction and fixation device can include a first unit, a second unit and a connection unit. The first unit has a first threaded portion and a first connecting portion. The second unit has a second threaded portion and a second connecting portion.

FIG. 11 illustrates a flowchart of the arthritis correction and fixation device operating method in this embodiment.

As shown in FIG. 11, the arthritis correction and fixation device operating method can include the following steps of:

Step 1010: connecting the first connecting portion and the second connecting portion through the connection unit, so that the first unit and the second unit can actuate relative to each other;

Step 1030: connecting the first unit with the distal phalange and connecting the second unit with the middle phalange; and

Step 1050: when the first threaded portion is rotated, the first connecting portion driving the second unit to move together in a direction away from the first threaded portion, thereby changing a distance between the distal phalange and the middle phalange.

In practical applications, the first unit can further include a screw disposed in the first threaded portion and engaged with the first connecting portion, and rotating directions of the screw and the first threaded portion are opposite to each other. When the first threaded portion is rotated in a first rotational direction (e.g., the clockwise direction), the screw rotates in a second rotational direction (e.g., the counterclockwise direction) opposite to the first rotational direction to move away from the first threaded portion, and the screw drives the first connecting portion engaged with the screw and the second unit connected to the first connecting portion both move away from the first threaded portion, thereby the distance between the distal phalange and the middle phalange can be increased through the joint distraction method. Therefore, the distal phalange and the middle phalange do not rub against each other, so it can effectively alleviate the pain caused by finger arthritis.

In practical applications, the first connecting portion has a first through hole and the second connecting portion has a second through hole. The first connecting portion can have an accommodating space for accommodating the second connecting portion. When the second connecting portion is accommodated in the accommodating space of the first connecting portion, the first through hole and the second through hole are aligned with each other, and the connection unit passes through the first through hole and the second through hole to connect the first connecting portion and the second connecting portion, so that the first unit and the second unit can relative to each other, but not limited to this.

Compared to the prior art, when the patient's finger is suffering from arthritis, the arthritis correction and fixation device of the invention can be disposed in the patient's finger and connected between the distal phalange and the middle phalange, and the joint distraction method can be used according to the actual needs of the patient to adjust the distance between the distal phalange and the middle phalange, so that the distal phalange and the middle phalange can be relatively open to prevent the distal phalange and the middle phalange from rubbing against each other, and the pain caused by the patient's finger movement can be effectively reduced.

With the example and explanations above, the features and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

What is claimed is:

1. An arthritis correction and fixation device, used to connect a distal phalange and a middle phalange of a finger, the arthritis correction and fixation device comprising:
a first unit connecting with the distal phalange and having a first threaded portion and a first connecting portion;
a second unit connecting with the middle phalange and having a second threaded portion and a second connecting portion; and
a connection unit connecting the first connecting portion and the second connecting portion, so that the first unit and the second unit can actuate relative to each other;
wherein when the first threaded portion is rotated, the first connecting portion drives the second unit to move together in a direction away from the first threaded portion, thereby changing a distance between the distal phalange and the middle phalange;
the first unit further comprises a screw disposed in the first threaded portion and engaged with the first connecting portion, and rotating directions of the screw and the first threaded portion are opposite to each other;
when the first threaded portion is rotated in a first rotational direction, the screw rotates in a second rotational direction opposite to the first rotational direction to move away from the first threaded portion, and the screw drives the first connecting portion engaged with the screw and the second unit connected to the first connecting portion both move away from the first threaded portion, thereby increasing the distance between the distal phalange and the middle phalange.

2. The arthritis correction and fixation device of claim 1, wherein the first connecting portion has an accommodating space for accommodating the second connecting portion.

3. The arthritis correction and fixation device of claim 2, wherein the first connecting portion has a first through hole and the second connecting portion has a second through hole; when the second connecting portion is accommodated in the accommodating space of the first connecting portion, the first through hole and the second through hole are aligned with each other, and the connection unit passes through the first through hole and the second through hole to connect the first connecting portion and the second connecting portion.
